(19) United States
(12) Patent Application Publication
(10) Pub. No.: US 2022/0136015 A1
(43) Pub. Date: May 5, 2022

(54) METHOD FOR PRODUCING OXYGEN ADDUCT OF SESQUITERPENE,COMPOUND AND COMPOSITION

(71) Applicant: T. HASEGAWA CO., LTD., Tokyo (JP)

(72) Inventors: Satoru UMEZAWA, Nakahara-ku, Kawasaki-shi, Kanagawa (JP); Shunsuke KONISHI, Nakahara-ku, Kawasaki-shi, Kanagawa (JP); Kuniki KINO, Tokyo (JP)

(21) Appl. No.: 17/435,146

(22) PCT Filed: Feb. 21, 2020
(86) PCT No.: PCT/JP2020/007082

§ 371 Date: Aug. 31, 2021

(30) Foreign Application Priority Data
Mar. 1, 2019 (JP) 2019-037136

(51) Int. Cl. C12P 7/26

(52) U.S. Cl. CPC C12P 7/26; C12Y 116/01007

(57) ABSTRACT

A method for producing an oxygen adduct of sesquiterpene, comprising: a step of providing a system comprising an iron reduction unit; a step of adding a trivalent iron compound to the system; and an oxygen addition step of performing oxygen addition to sesquiterpene in the system comprising the iron reduction unit and the trivalent iron compound is a novel method that can efficiently produce an oxygen adduct of sesquiterpene.

### TECHNICAL FIELD

The present invention relates to a method for producing an oxygen adduct of sesquiterpene, and a compound produced by this production method, and a composition.

### BACKGROUND ART

It is known that sesquiterpene can be used as an aroma compound (or a flavor or fragrance compound), and it is further known that an oxide of sesquiterpene may be used as an aroma compound (see Patent Literatures 1 to 3).

For example, Patent Literature 1 states that natural (−)-rotundone ((−)-(3S,5R,8S)-3,8-dimethyl-5-(prop-1-en-2-yl)-3,4,5,6,7,8-hexahydroazulen-1(2H)-one; hereinafter, also simply referred to as “rotundone”), an oxygen adduct of α-guaiene, is contained in Shiraz grape, black pepper and white pepper, etc. and can be used as an aroma composition (or a flavor or fragrance composition) in combination with rotundol. Patent Literature 1 describes a method for producing rotundone from α-guaiene, comprising (1) a step of allowing cytochrome P450 to act on α-guaiene to oxidize methylene at position 3 of the α-guaiene into carbonyl; and/or (2) a step of allowing cytochrome P450 to act on α-guaiene in the presence of an electron transfer protein having electron transferring activity against cytochrome P450 to oxidize methylene at position 3 of the α-guaiene into carbonyl, in order to produce rotundone with high yields and high purity.

Patent Literature 2 describes a method comprising reacting laccase with a material comprising α-guaiene and/or α-bulnesene (patchouli oil, etc.) in the presence of an oxygen source to produce a hydroxy group adduct thereof (rotundol in which a carbonyl group at position 1 of rotundone is an alcohol, etc.). Patent Literature 2 further states that: a mixture of many α-guaiene oxides including rotundone (see examples 3, 4 and 5) is obtained as by-products of the reaction; and this mixture retains floral or woody odor, etc.

Patent Literature 3 states that many guaiene oxides including rotundone are obtained through distillation, heating to reflux, redistillation, etc. of a guaiene-rich raw oil or the like.

### CITATION LIST

### Patent Literature

Patent Literature 1: Japanese Patent Laid-Open No. 2017-216974
Patent Literature 2: Japanese Translation of PCT International Application Publication No. 2013-534927
Patent Literature 3: International Publication No. WO 2018/153499
Patent Literature 4: Japanese Translation of PCT International Application Publication No. 2016-530264

### SUMMARY OF INVENTION

### Technical Problem

A method described in Examples of Patent Literature 1, however, converts 5 mM α-guaiene into 0.41 mM rotundone through microbial cell reaction for 17 hours using recombinant E. coli highly expressing cytochrome P450. Thus, for this method, further improvement in yield and reaction rate has been required. Patent Literature 1 states that no rotundone was detectable from 5 mM α-guaiene in microbial cell reaction at 30° C. for 17 hours using recombinant E. coli harboring an empty vector as a control.

Methods described in Patent Literatures 2 and 3 are not originally aimed at producing an oxygen adduct of sesquiterpene and result in various by-products. Therefore, for these methods, improvement in the yield and purity of an oxygen adduct of sesquiterpene have been required.

Patent Literature 4 states that α-pinene which is monoterpene is converted into p-cymene by isomerization and dehydrogenation, but does not describe application to sesquiterpene or performing oxidation to the point of oxygen addition instead of dehydrogenation.

An object of the present invention is to provide a method for producing an oxygen adduct of sesquiterpene which is a novel method that can efficiently produce an oxygen adduct of sesquiterpene.

### Solution to Problem

The present inventors have conducted diligent studies to attain the object and consequently found that an oxygen adduct of sesquiterpene can be efficiently produced through a step of intendedly adding a trivalent iron compound (oxidation number: +3; also referred to as Fe(III) or ferric iron) into a system comprising an iron reduction unit, reaching the completion of the present invention.

The configuration of the present invention which is a specific approach to attain the object, and a preferred configuration of the present invention will be described below.

[1] A method for producing an oxygen adduct of sesquiterpene, comprising:

a step of providing a system comprising an iron reduction unit;

a step of adding a trivalent iron compound to the system; and

an oxygen addition step of performing oxygen addition to sesquiterpene in the system comprising the iron reduction unit and the trivalent iron compound.

[2] The method according to [1], wherein the iron reduction unit is iron reductase.

[3] The method for producing an oxygen adduct of sesquiterpene according to [1] or [2], wherein the oxygen addition step is a step of selectively performing oxygen addition at an allyl position or a double bond of the sesquiterpene.

[4] The method for producing an oxygen adduct of sesquiterpene according to any one of [1] to [3], wherein

the sesquiterpene is α-guaiene, and

the oxygen adduct of sesquiterpene includes (−)-rotundone.

[5] The method for producing an oxygen adduct of sesquiterpene according to any one of [1] to [4], wherein the oxygen addition step is a step of selectively performing demethylation and oxygen addition at an allyl position of the sesquiterpene.

[6] The method for producing an oxygen adduct of sesquiterpene according to any one of [1] to [5], wherein

the sesquiterpene is α-guaiene,

the oxygen adduct of sesquiterpene includes (−)-rotundone and a demethylated isomer, and

the demethylated isomer is a compound represented by the following formula (1):

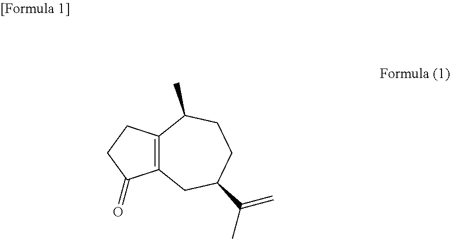

[7] The method for producing an oxygen adduct of sesquiterpene according to any one of [1] to [6], wherein a surfactant is added in the oxygen addition step or prior thereto.

[8] The method for producing an oxygen adduct of sesquiterpene according to any one of [1] to [7], wherein

cyclodextrin is added in the oxygen addition step or prior thereto, and

the cyclodextrin includes 2-hydroxypropyl-γ-cyclodextrin.

[9] The method for producing an oxygen adduct of sesquiterpene according to any one of [1] to [7], wherein

cyclodextrin is added in the oxygen addition step or prior thereto, and

the cyclodextrin includes at least one selected from α-cyclodextrin, β-cyclodextrin, γ-cyclodextrin and 2-hydroxypropyl-β-cyclodextrin.

[10] The method for producing an oxygen adduct of sesquiterpene according to any one of [1] to [9], wherein

the oxygen addition step is performed through microbial cell reaction, and

the iron reduction unit is iron reductase derived from the microbial cell.

[11] The method for producing an oxygen adduct of sesquiterpene according to [10], wherein the step of adding the trivalent iron compound to the system is performed before addition of the sesquiterpene.

[12] The method for producing an oxygen adduct of sesquiterpene according to [10] or [11], further comprising

a step of culturing the microbial cell, wherein

the trivalent iron compound is added in the step of culturing so that the trivalent iron compound is contained in the microbial cell.

[13] The method for producing an oxygen adduct of sesquiterpene according to [12], wherein the trivalent iron compound is added at any time from 6 hours before the completion of culture to the completion of culture.

[14] The method for producing an oxygen adduct of sesquiterpene according to [12] or [13], wherein the trivalent iron compound is added at 0.7 to 50 mM in the step of culturing.

[15] The method for producing an oxygen adduct of sesquiterpene according to any one of [10] to [14], wherein the microbial cell is non-recombinant E. coli.

[16] The method for producing an oxygen adduct of sesquiterpene according to any one of [10] to [14], wherein the microbial cell is a recombinant highly expressing at least one selected from the following (a) to (c):

(a) a protein having an amino acid sequence represented by any of SEQ ID NOs: 1 to 4;
(b) a protein which has an amino acid sequence having 90% or higher sequence identity to an amino acid sequence represented by any of SEQ ID NOs: 1 to 4, and has reductive activity against the trivalent iron compound; and
(c) a protein which has an amino acid sequence having 80% or higher sequence identity to an amino acid sequence represented by any of SEQ ID NOs: 1 to 4 with the conservative substitution of one or more amino acids from the amino acid sequence, and has reductive activity against the trivalent iron compound.

[17] The method for producing an oxygen adduct of sesquiterpene according to any one of [1] to [9], wherein the oxygen addition is performed through microbial cell-free reaction.

[18] The method for producing an oxygen adduct of sesquiterpene according to [17], wherein the iron reduction unit is iron reductase, and the oxygen addition is performed using a purified enzyme of the iron reductase.

[19] The method for producing an oxygen adduct of sesquiterpene according to [17] or [18], wherein the trivalent iron compound is added in the oxygen addition step.

[20] The method for producing an oxygen adduct of sesquiterpene according to [18] or [19], wherein the iron reductase is at least one selected from the following (a) to (c):

(a) a protein having an amino acid sequence represented by any of SEQ ID NOs: 1 to 4;
(b) a protein which has an amino acid sequence having 90% or higher sequence identity to an amino acid sequence represented by any of SEQ ID NOs: 1 to 4, and has reductive activity against the trivalent iron compound; and
(c) a protein which has an amino acid sequence having 80% or higher sequence identity to an amino acid sequence represented by any of SEQ ID NOs: 1 to 4 with the conservative substitution of one or more amino acids from the amino acid sequence, and has reductive activity against the trivalent iron compound.

[21] The method for producing an oxygen adduct of sesquiterpene according to any one of [2] to [20], wherein a coenzyme of the iron reductase is added in the oxygen addition step.

[22] The method for producing an oxygen adduct of sesquiterpene according to [21], wherein a regeneration system of the coenzyme is added in the oxygen addition step to regenerate the coenzyme.

[23] A compound represented by the following formula (1):

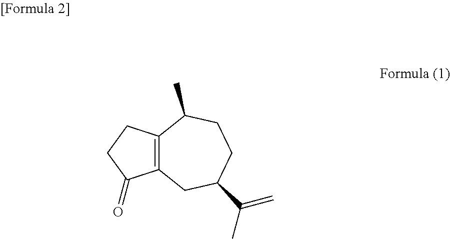

[24] A composition comprising (−)-rotundone and a compound according to [23].

### Advantageous Effects of Invention

The present invention can provide a method for producing an oxygen adduct of sesquiterpene which is a novel method that can efficiently produce an oxygen adduct of sesquiterpene.

### BRIEF DESCRIPTION OF DRAWINGS

FIG. 1 is a graph showing the concentrations of rotundone and a demethylated isomer in reaction solution extracts obtained through microbial cell reaction using E. coli in Examples 1 to 6 and Comparative Examples 1 to 3.

FIG. 2 is a chromatogram obtained by GC/MS analysis in Comparative Example 2.

FIG. 3 is a chromatogram obtained by GC/MS analysis in Example 4.

FIG. 4 is a graph showing the concentrations of rotundone and a demethylated isomer in reaction solution extracts obtained through purified enzyme reaction in Examples 101 to 104.

### DESCRIPTION OF EMBODIMENTS

Hereinafter, the present invention will be described in detail. Although components described below may be explained on the basis of typical embodiments and specific examples, the present invention is not limited by such embodiments. In the present specification, a numerical range indicated by the term “to” means that numerical values described before and after the term “to” are included as the lower limit value and the upper limit value.

[Method for Producing Oxygen Adduct of Sesquiterpene]

The method for producing an oxygen adduct of sesquiterpene according to the present invention comprises: a step of providing a system comprising an iron reduction unit; a step of adding a trivalent iron compound to the system; and an oxygen addition step of performing oxygen addition to sesquiterpene in the system comprising the iron reduction unit and the trivalent iron compound.

This configuration can efficiently produce an oxygen adduct of sesquiterpene.

Hereinafter, preferred aspects of the present invention will be described.

<Step of Providing System Comprising Iron Reduction Unit>

The iron reduction unit can be any iron reduction unit that can reduce the trivalent iron compound to form a divalent iron compound. The iron reduction unit may be, for example, an electrode capable of reducing the trivalent iron compound, or may be iron reductase. The system comprising the iron reduction unit can be an environment in which such an iron reduction unit exerts its ability to reduce iron.

(Electrode for Iron Reduction)

When the iron reduction unit is an electrode, the electrode that reduces the trivalent iron compound may be constituted by, for example, platinum. The system comprising the iron reduction unit can comprise a water tank, an aqueous solution containing an electrolyte such as a trivalent iron ion, a reference electrode, a working electrode (electrode that performs iron reduction), a counter electrode, an anion-exchange membrane, etc., and can be constructed with reference to, for example, “Report of Central Research Institute of Electric Power Industry, Electrochemical control of bacteria (Part 1)—Electrochemical cultivation of Thiobacillus ferrooxidans using soluble iron as an electron mediator—Research report: U95003” (Central Research Institute of Electric Power Industry, June 1995). When the iron reduction unit is an electrode for iron reduction, this electrode may be used in microbial cell-free reaction or may be used in microbial cell reaction, as mentioned later.

(Iron Reductase)

—Activity—

The iron reductase is not particularly limited as long as the iron reductase has activity of reducing the trivalent iron compound (reductive activity against the trivalent iron compound). Among others, it is preferred to have reductive activity against an iron chelate complex which is an organic iron compound, and it is preferred that the reduced iron, for example, a divalent iron compound, have activity of regioselectively performing oxygen addition. For example, it is preferred to have activity of selectively performing oxygen addition at an allyl position and/or a double bond (double bond position, preferably both the carbon atoms constituting the double bond) of the sesquiterpene. The iron reductase preferably reduces the trivalent iron compound so that the reduced iron has activity of selectively performing oxygen addition at an allyl position of the sesquiterpene.

The number of oxygen atoms added per sesquiterpene molecule is not particularly limited, and preferably, one oxygen atom is added per sesquiterpene molecule.

In the oxygen addition step, oxygen addition may be performed directly to the sesquiterpene, or the sesquiterpene may be subjected to another reaction and then oxygen addition. Examples of another reaction can include demethylation. Specifically, the iron reductase preferably also has activity of forming reduced iron as mentioned above so that the reduced iron demethylates the sesquiterpene. In an exemplary preferred aspect of the present invention, the oxygen addition step is preferably a step of selectively performing demethylation and oxygen addition at an allyl position of the sesquiterpene.

—Type of Iron Reductase—

Wild-type iron reductase may be used as the iron reductase, and the iron reductase can comprise the amino acid sequence of the wild-type iron reductase (wild-type amino acid sequence).

Specific examples of the wild-type iron reductase include, but are not limited to, Fre (Escherichia coli BL21 (DE3): ECD_03735; SEQ ID NO: 1), YqjH (Escherichia coli BL21 (DE3): ECD_02939; SEQ ID NO: 2), Fpr (Escherichia coli BL21 (DE3): ECD_03809; SEQ ID NO: 3), and NfsB (Escherichia coli BL21 (DE3): ECD_00539; SEQ ID NO: 4).

Among them, Fre is NAD(P)H flavin reductase and has activity of reducing free flavin serving as a coenzyme so that the reduced free flavin reduces the trivalent iron compound. Thus, in the case of using Fre, it is necessary to add flavin to a reaction system. By contrast, YqjH, Fpr, or NfsB is NAD(P)H ferric-chelate reductase and directly reduces the trivalent iron compound or a chelate complex thereof without the need of adding flavin because these enzymes contain flavin. In the case of using YqjH, Fpr, or NfsB, it is preferred to use an organic iron compound (chelate complex) such as Fe(III)-EDTA or Fe(III)-dicitrate as the trivalent iron compound in combination therewith.

The amino acid sequence of the iron reductase used in the present invention may comprise an amino acid sequence derived from the wild-type amino acid sequence by the mutation (i.e., deletion, substitution, and/or addition) of one or more amino acids as long as the iron reductase does not lose reductive activity against the trivalent iron compound. The mutation can produce any effect and may adjust, i.e., improve or reduce, reductive activity against the trivalent iron compound, for example. This adjustment can adjust the oxygen adding activity of the obtained reduced iron against the sesquiterpene.

In the present invention, the iron reductase is preferably at least one selected from the following (a) to (c):

(a) a protein having an amino acid sequence represented by any of SEQ ID NOs: 1 to 4;
(b) a protein which has an amino acid sequence having 90% or higher sequence identity to an amino acid sequence represented by any of SEQ ID NOs: 1 to 4, and has reductive activity against the trivalent iron compound; and
(c) a protein which has an amino acid sequence having 80% or higher sequence identity to an amino acid sequence represented by any of SEQ ID NOs: 1 to 4 with the conservative substitution of one or more amino acids from the amino acid sequence, and has reductive activity against the trivalent iron compound.

Hereinafter, (b) and (c) will be described in detail.

The number of amino acid mutations can be any number of one or more and is, for example, 30 or less, 20 or less, 15 or less, 10 or less, or several or less. The term “several” means 2, 3, 4, 5, 6, 7, 8, or 9.

Preferred examples of the amino acid mutation include conservative substitution. The conservative substitution is the replacement of an amino acid residue with a residue similar in physicochemical property and/or structure thereto. Substitution that works as the conservative substitution is known in the art about each amino acid, and as an example, corresponds to substitution between amino acids having the same polarity (basicity, acidity, or neutrality), charge, hydrophilicity, and/or hydrophobicity, substitution between aromatic amino acids or aliphatic amino acids, etc. More specifically, examples thereof include, but are not limited to, the substitution of Ala with Ser or Thr, the substitution of Arg with Gln, His or Lys, the substitution of Asn with Glu, Gln, Lys, His or Asp, the substitution of Asp with Asn, Glu or Gln, the substitution of Cys with Ser or Ala, the substitution of Gln with Asn, Glu, Lys, His, Asp or Arg, the substitution of Glu with Asn, Gln, Lys or Asp, the substitution of Gly with Pro, the substitution of His with Asn, Lys, Gln, Arg or Tyr, the substitution of Ile with Leu, Met, Val or Phe, the substitution of Leu with Ile, Met, Val or Phe, the substitution of Lys with Asn, Glu, Gln, His or Arg, the substitution of Met with Ile, Leu, Val or Phe, the substitution of Phe with Trp, Tyr, Met, Ile or Leu, the substitution of Ser with Thr or Ala, the substitution of Thr with Ser or Ala, the substitution of Trp with Phe or Tyr, the substitution of Tyr with His, Phe or Trp, and the substitution of Val with Met, Ile or Leu.

The position of the amino acid mutation can be any position as long as the iron reductase does not lose reductive activity against the trivalent iron compound. The position can be discussed on the basis of, for example, the three-dimensional structure of the iron reductase to be mutated.

The amino acid sequence of the iron reductase preferably has 70% or higher, 80% or higher, 85% or higher, 90% or higher, 95% or higher, 97% or higher, 98% or higher, or 99% or higher sequence identity to the wild-type amino acid sequence, for example, the amino acid sequence represented by SEQ ID NO: 1, 2, 3 or 4. As well known in the art, it is highly probable that an amino acid sequence having a given level of or higher identity has activity and substrate specificity similar to those of the unmutated protein.

When the amino acid mutation is conservative mutation, activity similar to that of the unmutated protein is easily maintained even if the identity of an amino acid sequence is relatively low. Therefore, in the case of conservative mutation, the sequence identity to the wild-type amino acid sequence, for example, the amino acid sequence represented by SEQ ID NO: 1, 2, 3 or 4, can be 60% or higher, 70% or higher, 80% or higher, 85% or higher, or 90% or higher.

The amino acid sequence identity can be confirmed by a method known in the art. Specific examples thereof include methods of calculating sequence identity using identity search software such as FASTA, BLAST, BLASTX, or Smith-Waterman [Meth. Enzym., 164, 765 (1988)] and default (initial setting) parameters.

The reductive activity of the iron reductase against the trivalent iron compound can have any strength. The activity can be, for example, 5% or higher, 10% or higher, 30% or higher, 50% or higher, 70% or higher, 90% or higher, 100% or higher, 120% or higher, or 150% or higher, with respect to the activity of any iron reductase described in Examples. The strength of the activity can be determined by any method and can be determined, for example, by comparing the yield of an oxygen adduct of sesquiterpene such as rotundone with a yield obtained using particular iron reductase described in Examples.

—Aspect of Step of Providing System Comprising Iron Reductase—

The system comprising the iron reductase can be provided using an organism or a cell producing the iron reductase, or a disruption product or a homogenate thereof. On the other hand, the system comprising the iron reductase can be provided using a purified enzyme of the iron reductase. The purified enzyme may be commercially obtained, or the purified enzyme may be collected by a method mentioned later from an organism or a cell producing the iron reductase, or a disruption product or homogenate thereof.

The organism or the cell producing the iron reductase can be appropriately cultured and thereby allowed to produce iron reductase. For the culture, preculture may be performed in order to increase the amount of microbial cells in a small amount of a medium, and then, the amount of the medium can be increased, followed by culture for sufficiently producing iron reductase.

The organism or the cell producing the iron reductase can be any organism or cell that can express iron reductase. The organism or the cell may naturally produce iron reductase, or does not naturally produce iron reductase but may be modified so as to produce iron reductase by a genetic modification technique or the like. For example, in the production method of the present invention, a cell of a microorganism or the like expressing wild-type trivalent iron compound reductase or previously listed trivalent iron compound reductase having a modified amino acid sequence may be used. In this respect, a cell highly expressing iron reductase gene through the adjustment of gene expression by a gene recombination technique, a RNA interference technique, a genome editing technique, an epigenome editing technique, or the like may be used.

Examples thereof include, but are not limited to, bacteria (E. coli, lactic acid bacteria, actinomycete, and Bacillus subtilis), yeasts, fungi, plants, plant cells, animal cells, and insect cells. In the present invention, it is preferred to use a microbial cell as the organism or the cell producing the iron reductase.

<Step of Adding Trivalent Iron Compound to System>

The type of the trivalent iron compound to be added is not particularly limited. The trivalent iron compound also includes a complex containing a trivalent iron ion. Examples of the trivalent iron compound can include inorganic iron compounds such as FeCl₃, and organic iron compounds. The trivalent iron compound is preferably an organic iron compound. The organic iron compound is preferably a complex. Examples thereof include Fe(III)-dicitrate, Fe(III)-tricatechol, Fe(III)-Tridopa, Fe(III)-EDTA, Fe(III)-NTA, Fe(III)-HEDTA, Fe(III)-DTPA, Fe(III)-tri-o-phenanthroline, and Fe(III)-TPA. EDTA represents ethylenediaminetetraacetic acid, NTA represents nitrilotriacetic acid, HEDTA represents (2-hydroxyethyl)ethylenediaminetriacetic acid, DTPA represents diethylenetriaminepentaacetic acid, and TPA represents tris(2-pyridylmethyl)amine.

A chelate complex having two or more ligands is more preferred, and a chelate complex having 4 or 5 coordination positions is more preferred. The coordinating atom is not particularly limited. The coordinating atom may be, for example, an oxygen atom, may be an oxygen atom and a nitrogen atom, or may be a nitrogen atom, and is preferably an oxygen atom and a nitrogen atom, or a nitrogen atom.

It is particularly preferred to use Fe(III)-EDTA, Fe(III)—NTA, Fe(III)—HEDTA, Fe(III)-DTPA, Fe(III)-tri-o-phenanthroline, or Fe(III)-TPA.

The timing of the step of adding a trivalent iron compound to the system is not particularly limited. The step of adding a trivalent iron compound to the system may be performed, for example, prior to the oxygen addition step, or may be performed at or after the start of the oxygen addition step.

In the case of performing oxygen addition (oxygen addition reaction) through microbial cell reaction mentioned later, it is preferred to perform the step of adding a trivalent iron compound to the system prior to the oxygen addition step. The details of this case will be described in description about microbial cell reaction mentioned later.

On the other hand, in the case of performing oxygen addition through microbial cell-free reaction mentioned later, it is preferred to perform the step of adding a trivalent iron compound to the system at and/or after the start of the oxygen addition step.

<Oxygen Addition Step>

The oxygen addition step involves performing oxygen addition to sesquiterpene in the system comprising the iron reduction unit and the trivalent iron compound, for example, a reaction system obtained by adding the trivalent iron compound into the system comprising the iron reductase.

(Sesquiterpene)

Examples of the sesquiterpene can include α-guaiene, α-bulnesene, rotundol, valencene, cedrene, caryophyllene, longifolene, zingiberene, cadinene, humulene, farnesol, patchoulol, α-bisabolene, β-bisabolene, and mixtures of two or more thereof.

The sesquiterpene is preferably α-guaiene, rotundol, (+)-valencene, β-caryophyllene, or (+)-longifolene, more preferably α-guaiene.

A method for obtaining such sesquiterpene is not particularly limited. The sesquiterpene used may be obtained from a plant by extraction, distillation, or the like and appropriately purified, or may be obtained as a commercially available product. For example, α-guaiene [(−)-(1S,4S,7R)-1,4-dimethyl-7-(prop-1-en-2-yl)-1,2,3,4,5,6,7,8-octahydroazulene] represented by the following formula (2) used may be obtained from a plant such as Guaiacum officinale by extraction, distillation, or the like and appropriately purified, or may be obtained as a commercially available product.

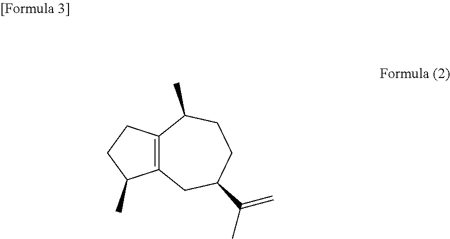

(Oxygen Adduct of Sesquiterpene)

The oxygen adduct of sesquiterpene is preferably an oxygen adduct of α-guaiene, rotundol, (+)-valencene, β-caryophyllene, (+)-longifolene, α-bisabolene, or β-bisabolene, more preferably an oxygen adduct of α-guaiene, rotundol, (+)-valencene, β-caryophyllene, or (+)-longifolene.

The oxygen adduct of β-caryophyllene or the (+)-longifolene is particularly preferably an oxygen adduct obtained by adding oxygen to a double bond of β-caryophyllene or the (+)-longifolene, particularly preferably epoxide cyclized by oxygen addition to a double bond thereof. The oxygen adduct of α-guaiene, the rotundol, the (+)-valencene, the α-bisabolene, or the β-bisabolene is particularly preferably an oxygen adduct of an allyl position thereof, particularly preferably an oxygen adduct of an allyl position (methylene at position 3) of α-guaiene.

The oxygen adduct obtained by adding oxygen to an allyl position of α-guaiene preferably comprises rotundone represented by the formula (3) given below, and is more preferably rotundone or a demethylated isomer thereof. The demethylated isomer is preferably a compound represented by the formula (1) given below.

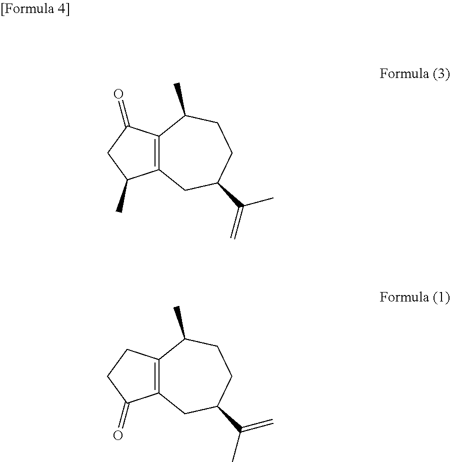 [SMILES: C=C(C)[C@@H]1CC[C@H](C)C2=C(C1)[C@@H](C)CC2=O ; C=C(C)[C@@H]1CC[C@H](C)C2=C(C1)C(=O)CC2]

Rotundone represented by the formula (3) is an important aroma component of black pepper and is an aroma compound characterizing the spicy odor of Shiraz wine. Rotundone has a threshold of its aroma at the lowest level among natural compounds. Rotundone is a compound known to have an enhancing effect on the flavor or aroma of various types of fruits, and is useful as an aroma compound. However, the content of rotundone in natural products is very low. Besides, an efficient synthesis method therefor has not yet been established. Rotundone is synthesized as one of many by-products by air oxidation or the like, or a genetically modified product (e.g., a recombinant microorganism) is used, while the yield is also low. The “genetic modification” means that a gene is intendedly engineered. The genetic modification includes, for example, gene recombination (transgenesis) in the narrow sense which transfers foreign genes, gene recombination in the broad sense including natural occurrence, cisgenesis (including self-cloning), and other approaches, mutagenesis such as point mutation, genome editing which edits the sequence of any target gene on the genome, and epigenome editing which edits the modification state of any target gene on the genome.

According to the present invention, rotundone can be efficiently produced. According to an exemplary preferred aspect of the present invention, rotundone can be produced at a high yield, high purity and a rapid reaction rate using a non-recombinant (specifically, non-recombinant E. coli harboring at least no foreign gene). Rotundone is suitable for industrial production and economically advantageous because α-guaiene easily available as a raw material is used as a starting material.

On the other hand, the compound represented by the formula (1) is a novel compound, and its details will be mentioned later.

—Aspect of Oxygen Addition Step—

In the oxygen addition step, the oxygen addition may be performed by mixing an organism or a cell producing the iron reductase, or a disruption product or a homogenate thereof or allowing it to coexist with a trivalent iron compound and sesquiterpene so that the iron reductase reduces the trivalent iron compound, and allowing the reduced iron (divalent iron compound) to act on the sesquiterpene, or the oxygen addition may be performed by allowing purified iron reductase to reduce a trivalent iron compound, and allowing the reduced iron (divalent iron compound) to act on sesquiterpene. The conditions for action can abide by conditions that are routinely used in methods for converting a given substrate to a biological conversion product using an organism or a cell itself having the enzyme activity of interest, or an isolated cell or a preparation (disruption product or homogenate), etc. thereof. For example, in the case of using live cells (or live microbial cells) expressing iron reductase, or live cells (or live microbial cells) carrying the protein, a trivalent iron compound and sesquiterpene can be allowed to coexist with each other in a medium that can culture these cells, etc., and incubated in an environment having no adverse effect on these cells, etc., for example, at a physiological temperature for a given time. On the other hand, in the case of using a reaction system comprising iron reductase (which may be a purified enzyme), or isolated cells or a preparation (disruption product or homogenate), etc. having iron reductase, the iron reductase, a trivalent iron compound and sesquiterpene can be mixed or allowed to coexist with each other and incubated for a given time in an aqueous solution buffered, if necessary, with a physiologically acceptable buffer. The incubation time can be determined with reference to, for example, the amount of the oxygen adduct of sesquiterpene converted from sesquiterpene, which can be determined by a method mentioned later using, if necessary, aliquots of the reaction mixture sampled over time.

In the oxygen addition step, it is preferred to use a microbial cell as the organism or the cell producing the iron reductase.

In the case of using microbial cells, the reaction system in the oxygen addition step is not particularly limited. The oxygen addition step using the iron reductase may be performed through microbial cell reaction or may be performed through microbial cell-free reaction.

Hereinafter, the case of performing the oxygen addition step using the iron reductase through microbial cell reaction, and the case of performing this step through microbial cell-free reaction will be separately described in order.

(1) Microbial Cell Reaction

The case of performing the oxygen addition step through microbial cell reaction (in vivo) will be described.

The oxygen addition step can be performed under conditions where the iron reduction unit, sesquiterpene, and microbial cells coexist with each other and where the microbial cells are capable of proliferating.

When the iron reduction unit is an electrode, this step can be carried out in a system comprising, as mentioned above, a water tank, a culture solution containing an electrolyte, a reference electrode, a working electrode (electrode that performs iron reduction; platinum, etc.), a counter electrode, an anion-exchange membrane, etc., and comprising microbial cells capable of proliferating therein.

When the iron reduction unit is iron reductase, the iron reductase is preferably derived from the microbial cell. The microbial cell reaction is preferably resting microbial cell reaction. The resting microbial cell reaction refers to reaction in which after culturing the microbial cells, a substrate (sesquiterpene according to the present invention) is added after the proliferation of cultured microbial cells is substantially arrested.

A genetically modified product may be used as the microbial cell, or a genetically unmodified product (microbial cell that has not undergone genetic modification; e.g., wild type or a natural mutant thereof) may be used. The genetic modification may be performed to the iron reductase gene, or may be modification to a gene or a base involved in the expression mechanism of the iron reductase gene.

In the present invention, it is preferred to use a non-recombinant. Specifically, it is preferred to use non-recombinant microbial cells or animal or plant cells expressing iron reductase. Examples of the non-recombinant that may be used include: (A) non-recombinants in the narrow sense which harbor no foreign gene but may have undergone natural occurrence, cisgenesis, mutagenesis, genome editing or epigenome editing; and (B) non-recombinants in the broad sense which neither harbor a foreign gene nor have undergone natural occurrence or cisgenesis but may have undergone mutagenesis, genome editing or epigenome editing.

In this case, it is preferred to use non-recombinant microbial cells or animal or plant cells that express iron complex reductase (ferric-chelate reductase) as the iron reductase and are capable of forming a complex of divalent iron. Such microbial cells or animal or plant cells are not particularly limited. The microbial cells are preferably bacteria. Examples thereof can include E. coli (Escherichia coli), yeasts (Saccharomyces cerevisiae), and denitrification bacteria (Paracoccus denitrificans). E. coli is particularly preferred. The animal or plant cells are preferably plant cells. Examples thereof can include Arabidopsis thaliana, peanut, beet, barley, rice, and tomato.

In the present invention, (C) a genetically unmodified product that has not undergone intended genetic modification may be used as the non-recombinant. Examples of the genetically unmodified product include products that neither harbor a foreign gene nor have undergone natural occurrence, cisgenesis, mutagenesis, genome editing or epigenome editing. Among the genetically unmodified products, wild type may be used, or a natural mutant may be used.

Alternatively, a genetically unmodified product or a non-recombinant that has not undergone the engineering of expression by a RNA interference technique may be used. Examples thereof include products harboring neither an expression vector for RNA for RNA interference nor a complex of RNA for RNA interference and a protein.

In the case of using recombinants of microbial cells, the microbial cells are not limited. The recombinant highly expressing iron reductase can be produced by any method and can be produced, for example, by expressibly inserting iron reductase gene to a vector to prepare a vector, and transferring the vector to microbial cells. A specific method is not particularly limited, and a known method can be used. As for the type of the microbial cells and the recombinant production method, for example, a method described in [0082] to [0091] of Japanese Patent Laid-Open No. 2017-216974 can be used as it is or by appropriate modification. The contents of the patent literature are incorporated herein by reference.

Wild-type iron reductase and its gene can be preferably used as the iron reductase and its gene.

In the case of using recombinants of microbial cells, the microbial cells are preferably recombinants highly expressing Fre, YqjH, Fpr or NfsB, more preferably recombinants highly expressing YqjH, Fpr or NfsB, particularly preferably recombinants highly expressing YqjH or Fpr.

In the case of performing the oxygen addition step through microbial cell reaction, the step of adding a trivalent iron compound to the system is preferably performed before addition of the sesquiterpene. Specifically, it is more preferred that the method further comprise a step of culturing the microbial cell prior to the oxygen addition step, wherein the trivalent iron compound is added in the step of culturing so that the trivalent iron compound is contained in the microbial cell, and the sesquiterpene is added in the oxygen addition step. It is preferred that the trivalent iron compound (preferably a trivalent iron chelate complex) is added at the time of culturing the microbial cell so that this compound accumulates intracellularly in the microbial cell, without adding the trivalent iron compound at the time of oxygen addition reaction. However, the step of adding a trivalent iron compound to the system and the oxygen addition step may be performed after the step of culturing of the microbial cell and addition of the sesquiterpene.

In the case of adding a trivalent iron compound in the step of culturing, it is preferred that the trivalent iron compound be added at any time from 6 hours before the completion of culture to the completion of culture (also referred to as a late stage of culture), from the viewpoint of elevating the yield of the oxygen adduct of sesquiterpene.

The production ratios of a plurality of oxygen adducts of sesquiterpene contained in the resulting reaction mixture can be adjusted by controlling the timing of addition of the trivalent iron compound in the step of culturing. For example, in the case of obtaining a reaction mixture of a non-demethylated isomer and a demethylated isomer as oxygen adducts of sesquiterpene, the production ratio of the non-demethylated isomer can be elevated by adding the trivalent iron compound at any time from the start of culture to 6 hours before the completion of culture (also referred to as an early stage of culture). In this case, the production ratios of the non-demethylated isomer and the demethylated isomer can be rendered equivalent by adding the trivalent iron compound at the late stage of culture.

In the case of adding a trivalent iron compound at the late stage of culture, it is preferred to add this compound at any time from 4 hours before the completion of culture to the completion of culture, and it is more preferred to add the compound at any time from 3 hours before the completion of culture to the completion of culture. Specifically, when the culture time is, for example, 16 hours, it is preferred to add the compound about 13 hours after the start of culture, i.e., 3 hours before the completion of culture.

The concentration of the trivalent iron compound in the reaction system is not particularly limited and can be determined according to the amount of the microbial cell, the amount of the substrate, etc. within a range that allows the microbial cell to grow. The trivalent iron compound is preferably added at, for example, 0.7 to 50 mM, more preferably 1 to 30 mM, particularly preferably 1 to 15 mM, particularly preferably 3 to 10 mM (in terms of a final concentration), in the step of culturing.

In the case of performing the oxygen addition step through microbial cell reaction, examples of other components that may be added to the reaction system in the oxygen addition step can include carbon sources such as glucose, buffer solutions, and ethanol.

In the case of performing the oxygen addition step through microbial cell reaction, the reaction temperature is preferably 15 to 35° C., more preferably 20 to 30° C., particularly preferably 25 to 30° C.

In the case of performing the oxygen addition step through microbial cell reaction, the reaction time can be any reaction time and may be determined according to the desired amount of the product, etc. For example, even a reaction time of 2 to 16 hours, 4 to 10 hours, or 5 to 8 hours can produce an oxygen adduct of sesquiterpene, or the reaction may be performed for 24 hours or longer.

(2) Microbial Cell-Free Reaction

The case of performing the oxygen addition step through microbial cell-free reaction (in vitro) will be described.

When the iron reduction unit is an electrode, this step can be carried out in a system comprising, as mentioned above, a water tank, a culture solution containing an electrolyte, a reference electrode, a working electrode (electrode that performs iron reduction; platinum, etc.), a counter electrode, an anion-exchange membrane, etc.

When the iron reduction unit is iron reductase, the oxygen addition reaction is preferably performed using a purified enzyme of the iron reductase. A method for collecting the purified enzyme of the iron reductase is not particularly limited, and a known method can be used. For example, iron reductase present in cultures of the recombinant (including a culture supernatant and/or a cultured recombinant) obtained by the method for producing the recombinant highly expressing iron reductase mentioned above can be extracted and purified by known methods. The iron reductase of interest can be obtained using, for example, a solvent extraction method, a salting-out method, a solvent precipitation method, a dialysis method, an ultrafiltration method, gel electrophoresis, gel filtration chromatography, ion-exchange chromatography, reverse-phase chromatography, and affinity chromatography alone or in appropriate combination.

The iron reductase is preferably Fre, YqjH, Fpr or NfsB, more preferably YqjH, Fpr or NfsB, particularly preferably YqjH or Fpr.

In the case of performing the oxygen addition step through microbial cell-free reaction, the trivalent iron compound is preferably added in the oxygen addition step. In the case of adding a trivalent iron compound to a reaction system in the oxygen addition step, the concentration of the trivalent iron compound in the reaction system is not particularly limited and can be determined according to the amount of the substrate, etc. The trivalent iron compound is preferably added at, for example, 0.7 to 50 ml, more preferably 1 to 30 mM, particularly preferably 1 to 15 mM, particularly preferably 3 to 10 mM (in terms of a final concentration).

In the case of performing the oxygen addition step through microbial cell-free reaction, examples of other components that may be added to the reaction system in the oxygen addition step can include buffer solutions and ethanol.

In the case of performing the oxygen addition step through microbial cell-free reaction, the reaction temperature is preferably 15 to 35° C., more preferably 20 to 30° C.

In the case of performing the oxygen addition step through microbial cell-free reaction, the reaction time can be any reaction time and may be determined according to the desired amount of the product, etc. For example, even a reaction time of 1 to 16 hours, 2 to 10 hours, or 2 to 8 hours can produce an oxygen adduct of sesquiterpene, or the reaction may be performed for 24 hours or longer.

(Coenzyme of Iron Reductase and Regeneration System Thereof)

In both the cases of microbial cell reaction and microbial cell-free reaction, a coenzyme of the iron reductase may be present in the reaction system in the oxygen addition step. The coenzyme of the iron reductase is preferably added in the oxygen addition step. Examples of the coenzyme can include, but are not limited to, NADH (nicotinamide adenine dinucleotide) and NADPH (nicotinamide adenine dinucleotide phosphate). The timing of addition of the coenzyme to the system is not particularly limited. The coenzyme can be added in any step in the production method of the present invention or may be added prior to or during the oxygen addition step. The concentration of the coenzyme in the system is not particularly limited and can be in the range of 0.1 to 20 mM or 1 to 10 mM.

In this case, a compound constituting a regeneration system of the coenzyme may be further present in the reaction system in the oxygen addition step. The regeneration system of the coenzyme is preferably added in the oxygen addition step to regenerate the coenzyme. In the present specification, the regeneration system of the coenzyme includes a component involved in the regeneration of the coenzyme. Typical examples of such a component include glucose and glucose dehydrogenase, glycerol and glycerol dehydrogenase, and formic acid and formic acid dehydrogenase. Glucose and glucose dehydrogenase are preferably added in the oxygen addition step to regenerate NADH or NADPH.

Particularly, in the case of microbial cell-free reaction, it is preferred that the coenzyme be present in the reaction system, and it is more preferred that the coenzyme and the regeneration system of the coenzyme be present in the reaction system. In the case of microbial cell reaction, the coenzyme or the regeneration system of the coenzyme may or may not be present in the reaction system in the oxygen addition step, depending on the characteristics of the microbe used, the desired yield, etc. The concentration of the regeneration system of the coenzyme in the reaction system can be determined according to the coenzyme concentration, etc. and can be, for example, 0.01 to 10 U/ml.

(Surfactant)

For the microbial cell reaction or the microbial cell-free reaction, a surfactant may be present in the reaction system in the oxygen addition step. The surfactant is preferably added in the oxygen addition step or prior thereto. The surfactant can be added to the system at any timing, and may be added at any timing in the step of providing a system comprising iron reductase, the step of adding iron reductase, and the oxygen addition step or may be added between these steps. The surfactant is preferably added in an effective amount to the reaction system in the oxygen addition step, from the viewpoint of improving the yield of the oxygen adduct of sesquiterpene, i.e., the amount of the oxygen adduct of sesquiterpene formed.

The concentration of the surfactant used is preferably 0.01 to 1.0% by volume (v/v), more preferably 0.1 to 0.7% by volume, with respect to the volume of the reaction system.

The type of the surfactant can be any cationic surfactant, anionic surfactant, zwitterionic surfactant or nonionic surfactant. The surfactant can be arbitrarily selected according to the desired degree of protein solubilization. It is preferred to use a nonionic surfactant or a zwitterionic surfactant which is relatively less likely to denature proteins.

Specific examples of the cationic surfactant include sodium dodecyl sulfate (SDS).

Specific examples of the anionic surfactant include cetyl trimethyl ammonium bromide (CTAB).

Specific examples of the zwitterionic surfactant include CHAPS (3-[(3-cholamidopropyl)dimethylammonio]propanesulfonate) and CHAPSO (3-[(3-cholamidopropyl)dimethylammonio]-2-hydroxypropanesulfonate).

Specific examples of the nonionic surfactant include Tween® 20, Tween® 40, Tween® 80, PLURONICS™ F-127, polyethylene glycol (PEG), Triton X-100, Triton X-114, Brij-35, and Brij-58.

(Cyclodextrin)

For the microbial cell reaction or the microbial cell-free reaction, cyclodextrin may be added to the reaction system. The cyclodextrin is preferably added in the oxygen addition step or prior thereto. The cyclodextrin can be added to the system at any timing and may be added at any timing in the step of providing a system comprising iron reductase, the step of adding iron reductase, and the oxygen addition step or may be added between these steps. While not wishing to be bound by any theory, the substrate solubilizing effect of the cyclodextrin can improve the yield of the oxygen adduct of sesquiterpene, i.e., the amount of the oxygen adduct of sesquiterpene formed.

Examples of the cyclodextrin can include α-cyclodextrin (α-CD), β-cyclodextrin (β-CD), γ-cyclodextrin (γ-CD), 2-hydroxypropyl-β-cyclodextrin (HP-β-CD) and 2-hydroxypropyl-γ-cyclodextrin (HP-γ-CD). Among them, β-CD, γ-CD, HP-β-CD and HP-γ-CD are preferred, β-CD, HP-β-CD and HP-γ-CD are more preferred, and HP-β-CD and HP-γ-CD are particularly preferred. One cyclodextrin may be used singly, or two or more thereof may be used in combination. In the case of using two or more thereof in combination, two or more of the cyclodextrins mentioned above can be arbitrarily combined. Two or more members selected from β-CD, γ-CD, HP-β-CD and HP-γ-CD are preferred, and two or more members selected from β-CD, HP-β-CD and HP-γ-CD are more preferred. Two members HP-β-CD and HP-γ-CD are particularly preferably used in combination.

The concentration of the cyclodextrin is not particularly limited, and the cyclodextrin can be used in an amount that produces the effect of interest. Examples thereof can include the following numerical values.

In the case of using one type of cyclodextrin singly, the concentration can be 0.1% (w/v) or higher, 1% (w/v) or higher, 2% (w/v) or higher, 5% (w/v) or higher, 6% (w/v) or higher, 8% (w/v) or higher, 10% (w/v) or higher, 12% (w/v) or higher, 15% (w/v) or higher, 18% (w/v) or higher, 20% (w/v) or higher, 25% (w/v) or higher, or 30% (w/v) or higher. The concentration is preferably 1% (w/v) or higher, more preferably 2% (w/v) or higher, further preferably 5% (w/v) or higher, furthermore preferably 10% (w/v) or higher. The upper limit can be 50% (w/v), 40% (w/v), or 30% (w/v).

In the case of using two or more types of cyclodextrins in combination, the concentration can be, for example, 0.1% (w/v) or higher, 1% (w/v) or higher, 2% (w/v) or higher, 5% (w/v) or higher, 6% (w/v) or higher, 8% (w/v) or higher, 10% (w/v) or higher, 12% (w/v) or higher, 15% (w/v) or higher, 18% (w/v) or higher, 20% (w/v) or higher, 24% (w/v) or higher, 27% (w/v) or higher, 30% (w/v) or higher, or 40% (w/v) or higher, in terms of the total amount of the plural types of cyclodextrins. The concentration can be preferably 1% (w/v) or higher, more preferably 2% (w/v) or higher, further preferably 6% (w/v) or higher, furthermore preferably 10% (w/v) or higher, particularly preferably 20% (w/v) or higher. The upper limit can be 50% (w/v), 45% (w/v), 40% (w/v), 35% (w/v), or 30% (w/v).

In the case of using two or more types of cyclodextrins in combination, the mass ratio of each cyclodextrin can be any ratio and can be appropriately determined so as to produce the desired effect. As an example, two types HP-β-CD and HP-γ-CD are used in combination, and their mass ratios are in the range of 5:1 to 1:5, more preferably in the range of 4:1 to 1:4, further preferably in the range of 3:1 to 1:3, furthermore preferably in the range of 2:1 to 1:2, and can be, for example, 1:1.

<Purification and Isolation Step>

When a reaction mixture containing a plurality of oxygen adducts of sesquiterpene is obtained, the method for producing an oxygen adduct of sesquiterpene according to the present invention may comprise a step of purifying and isolating each oxygen adduct of sesquiterpene from the reaction mixture.

The purification and isolation step is not particularly limited, and a known method can be used. Each oxygen adduct can be purified and isolated by, for example, extraction, distillation, chromatography, crystallization, or fractional extraction.

[Compound Represented by Formula (1)]

The present invention also relates to a compound represented by the following formula (1):

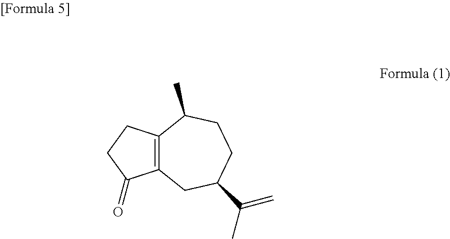

The compound represented by the formula (1) is a compound that takes on aroma. The compound represented by the formula (1) takes on musty and/or earthy odor in itself, and is capable of conferring characteristic flavor or aroma such as astringency, refreshing bitterness, or powdery feeling when blended in an appropriate amount alone or as an aroma composition containing it with various articles such as food or drink products, fragrance and cosmetic products, and other articles of taste. Examples of the purpose of the compound represented by the formula (1) include, but are not limited to, purposes of imparting aroma to food or drink products including coffee, burdock, and carrot, or food or drink products that take on their flavor, or fragrance and cosmetic products.

The compound represented by the formula (1) produced by the method for producing an oxygen adduct of sesquiterpene according to the present invention may be appropriately separated and purified from rotundone by a known technique, for example, silica gel chromatography or distillation.

[Composition]

The composition of the present invention comprises rotundone and a compound represented by the formula (1).

The composition of the present invention may be a reaction mixture containing a plurality of oxygen adducts of sesquiterpene produced by the method for producing an oxygen adduct of sesquiterpene according to the present invention.

The composition of the present invention comprising rotundone and the compound represented by the formula (1) can be used as a composition capable of imparting flavor or aroma that can be imparted by both or any one of the components to various articles (e.g., food or drink products, fragrance and cosmetic products, and other articles of taste), i.e., as an aroma composition, when blended with the articles.

In the composition of the present invention, for example, the concentration ratios of the rotundone and the compound represented by the formula (1) can be adjusted. For example, when the ratio of the rotundone is high, fresh feeling or juicy feeling is more sharply enhanced. When the ratio of the compound represented by the formula (1) is high, musty and/or earthy flavor or aroma is more sharply enhanced. When the concentration of the rotundone is high, spice-like, pepper-like and/or woody flavor or aroma may also be emphasized.

The composition comprising rotundone and the compound represented by the formula (1) as oxygen adducts of sesquiterpene produced by the method for producing an oxygen adduct of sesquiterpene according to the present invention can be blended as it is in any amount with various articles such as consumer goods (e.g., food or drink products and fragrance and cosmetic products) and other articles of taste, and can confer or enhance diverse flavor or aroma according to the concentration ratios of the rotundone and the compound represented by the formula (1). For example, fruit flavor or aroma can be enhanced. Particularly, fresh feeling or juicy feeling can be enhanced. The composition comprising rotundone and the compound represented by the formula (1) may be mixed with other components to prepare an aroma composition, and this aroma composition can be used to enhance, for example, fruit flavor or aroma, particularly, fresh feeling or juicy feeling, for consumer goods (e.g., food or drink products and fragrance and cosmetic products) and other articles of taste, etc.

[Aroma Composition]

<Component that May be Used in Combination with Rotundone and/or Compound Represented by Formula (1)>

Rotundone, the compound represented by the formula (1), or the composition of the present invention comprising both of them can be used as it is or as an aroma composition comprising it, as mentioned above. This aroma composition can be blended with various articles and thereby used to confer diverse flavor or aroma.

Examples of components that may be blended into such an aroma composition can include those described below.

Examples of other flavor or fragrance ingredients can include synthetic aroma compounds, natural essential oils, natural aroma compounds, and animal or plant extracts described in “Japan Patent Office, Collection of Well-known Prior Arts (flavor or fragrance ingredients) Part II, Flavorings, p. 8-87, issued on Jan. 14, 2000”.

The aroma composition can optionally contain solvents such as water and ethanol, and flavoring or fragrance fixatives such as ethylene glycol, 1,2-propylene glycol, glycerin, benzyl benzoate, triethyl citrate, Hercolyn, fatty acid triglyceride, and fatty acid diglyceride, which are usually used in aroma compositions.

The aroma composition can be prepared as an emulsified aroma composition using an emulsifier such as glycerin fatty acid ester, sorbitan fatty acid ester, propylene glycol fatty acid ester, sucrose fatty acid ester, lecithin, quillaia saponin, or casein sodium, and can be prepared as a powdered aroma composition by the addition of gum arabic or dextrin and drying.

The aroma composition can be added in any amount to various consumer goods (e.g., food or drink products and fragrance and cosmetic products) and other articles of taste, etc.

More specific examples of the fragrance and cosmetic products can include, but are not limited to: perfumes such as eau de cologne, eau de toilette, eau de parfum, and parfum; haircare products such as shampoo, rinse, and hair dressing (hair cream, pomade, etc.); cosmetics such as foundation, lipstick, lip cream, lip gloss, lotion, cosmetic emulsion, cosmetic cream, cosmetic gel, beauty essence, and pack; deodorant products such as antiperspirant spray, deodorant sheet, deodorant cream, and deodorant stick; inorganic salt, refreshing, carbon dioxide, skincare, enzymatic, or crude drug bathwater additives; cosmetics for sunburn such as suntan products and sunscreen products; facial cleansers such as soap for the face and facial cleansing cream; cleansers such as soap for the body or body soap, laundry soap, laundry detergents, detergents for disinfections, deodorant detergents, softeners, kitchen detergents, and detergents for cleanings; health and sanitary materials such as tooth brush, tissue paper, and toilet paper; aromatic deodorizers for spaces such as the inside of rooms or the inside of vehicles, various aromatic deodorizers for articles such as articles for daily use or furniture, and refreshers such as room fragrance; and noxious animal repellant insecticides such as insect deterrents, moth proofing agents, and insecticides. Consumer goods that are used in a form capable of attaching the aroma composition to articles such as fabrics, the skin, and hair is particularly preferred. In this context, the article is preferably an article having a fibrous structure on the surface. Preferred examples thereof include various fiber products such as towels, washcloth, cloth, bedclothes, curtains, carpets, and clothes.

More specific examples of the food or drink products include, but are not limited to: confectionery including rice confectionery such as rice cracker and rice cake, confectionery containing sweet bean paste, Uiro (Japanese steamed cake made of rice flour and sugar), Yokan (adzuki-bean jelly), jelly, baked or fried goods such as castella cake, biscuit, cooky, pie, cake, and chips, and pudding, cream, chocolate, gum, caramel candy, dip, spread, and paste; bread; noodles such as Udon (wheat noodle), buckwheat noodle, and ramen (Chinese noodle); cooked rice such as sushi, Gomoku-meshi (Japanese pilaf), fried rice and pilaf; Chinese foods such as jiao-zi (dumpling), shao mai (Chinese steamed dumplings), and spring roll; food made of flour such as Okonomiyaki (Japanese thin and flat pancake cooked on a hot plate with bits of meat, seafood and chopped cabbages) and Takoyaki (octopus dumpling); pickled foods and pickle mix; processed drink or food products of seafood; processed drink or food products of meat; seasonings such as salt, cooking salt, soy sauces, miso, sprinkle, seasoning for rice soup, margarine, mayonnaise, dressing, vinegars, seasoning sauces, sauces, ketchup, dipping sauces, curry roux, stew roux, soup roux, instant bouillon, composite seasonings, Mirin (sweet cooking rice wine)-like seasonings, and mix flour; dairy products such as cheese, yogurt, and butter; simmered foods such as simmered vegetables, Oden (Japanese fish cake stew), and pot stuff; takeout packed lunch ingredients and prepared foods; fruit juice, fruit juice beverages or fruit juice-containing refreshing beverages, and fruit beverages containing fruit pulp or granules; vegetable-containing food or drink products such as beverages and soups containing vegetables; drink products of taste such as sport drinks, honey beverages, nutritious supplement drinks, lactic acid bacteria beverages, coffee beverages, cocoa beverages, green tea, black tea, oolong tea, refreshing beverages, cola beverages, fruit juice beverages, milk beverages, and beer-flavored beverages; beverages containing crude drugs or herbs; and alcohol beverages such as wine, beer, Shochu-based beverages, spirit-based drinks, sparkling liquors, fruit liquors, flavored liquors, and other brews (sparkling) or liqueurs (sparkling) such as so-called “third beer”.

Examples of other articles of taste include, but are not limited to, cigarettes and electronic cigarettes.

<Rotundone and Compound Represented by Formula (1) in Aroma Composition>

The rotundone is capable of imparting spice-like, pepper-like, and/or woody flavor or aroma in itself to various articles. The rotundone can vary in flavor or aroma that may be conferred thereby, depending on a concentration. In the case of using a very small amount thereof in an article, the rotundone can confer or enhance, for example, fresh feeling or juicy feeling of fruits including citrus. The content of the rotundone in the aroma composition is not particularly limited. The content differs depending on components such as other flavor or fragrance ingredients to be mixed therewith, and cannot be generalized. Its concentration can usually be in the range of 0.5 ppt to 0.5 ppm, preferably 1 ppt to 0.2 ppm, with respect to the weight of the aroma composition.

The content of the rotundone in various consumer goods (e.g., food or drink products and fragrance and cosmetic products) in enhancing the flavor or aroma, particularly, fresh feeling or juicy feeling, of fruits differs depending on the types or forms of the consumer goods, and cannot be generalized. Its concentration for food or drink products can usually be in the range of 0.005 ppt to 5 ppt, preferably 0.01 ppt to 2 ppt, based on the weight of the food or drink products. The concentration for fragrance and cosmetic products can be in the range of 0.05 ppt to 0.5 ppt, preferably 0.1 ppt to 20 ppt, based on the weight of the fragrance and cosmetic products. For preferred purposes of the rotundone, see, for example, Japanese Patent Laid-Open No. 2016-198025 or 2016-198026 regarding food or drink products.

The compound represented by the formula (1) is capable of imparting musty and/or earthy flavor or aroma in itself to various articles. The concentration of the compound represented by the formula (1) in the aroma composition is not particularly limited. The concentration differs depending on components such as other flavor or fragrance ingredients to be mixed therewith, and cannot be generalized. Its concentration can usually be in the range of 0.01 ppt to 100 ppm, preferably 0.1 ppb to 1 ppm, based on the weight of the aroma composition.

The amount of the compound represented by the formula (1) used in various articles cannot be generally determined, as in the rotundone, and is not particularly limited. Its concentration for food or drink products can usually be in the range of 0.0001 ppt to 100 ppb, preferably 0.001 ppt to 100 ppt, based on the weight of the food or drink products. The concentration for fragrance and cosmetic products can usually be in the range of 0.001 ppt to 1 ppm, preferably 0.01 ppt to 10 ppb, based on the weight of the fragrance and cosmetic products.

### EXAMPLES

Hereinafter, features of the present invention will be described more specifically with reference to Examples and Comparative Examples. Materials, amounts of use, proportions, contents of treatment, treatment procedures, etc. described in Examples given below can be appropriately changed without departing from the spirit of the present invention. Thus, the scope of the present invention should not be interpreted as being limited by the specific examples given below.

### [Examples 1 to 6 and Comparative Examples 1 to 3]: Microbial Cell Reaction Using Non-Recombinant E. coli

<Preculture, Culture, and Preparation of Buffer Solution for Microbial Cell>

Non-recombinant E. coli was inoculated to a 5 ml scale of LB medium (containing 1′% (w/v) tryptone, 0.5% (w/v) yeast extracts, and 1% (w/v) NaCl; pH 7.0) and precultured at 37° C. for 8 hours.

Subsequently, 1 ml of the preculture solution was re-inoculated to a 100 ml scale of LB medium and cultured at 37° C. for 16 hours. Fe(III)-EDTA was added during the culture under any of the following conditions.

Culture condition 1. No addition of Fe(III)-EDTA.
Culture condition 2. Addition of Fe(III)-EDTA at 1 mM in terms of a final concentration 12 hours before the completion of culture (after a lapse of 4 hours from the start of culture after re-inoculation). The final concentration means the concentration of addition to the medium.
Culture condition 3. Addition of Fe(III)-EDTA at 5 mM in terms of a final concentration 12 hours before the completion of culture.
Culture condition 4. Addition of Fe(III)-EDTA at 1 mM in terms of a final concentration 3 hours before the completion of culture (after a lapse of 13 hours from the start of culture after re-inoculation).
Culture condition 5. Addition of Fe(III)-EDTA at 5 mM in terms of a final concentration 3 hours before the completion of culture.
Culture condition 6. Addition of Fe(III)-EDTA at 10 mM in terms of a final concentration 3 hours before the completion of culture.
Culture condition 7. Addition of Fe(III)-EDTA at 20 mM in terms of a final concentration 3 hours before the completion of culture.

The E. coli thus cultured was collected by centrifugation. The collected E. coli was suspended in a potassium phosphate buffer solution (50 mM, pH 7.5) to obtain a potassium phosphate buffer solution of the E. coli.

E. coli harboring an empty vector, i.e., a pET21a plasmid or a pMW218 plasmid having no insert of any gene, as described in [0119] of Japanese Patent Laid-Open No. 2017-216974 (hereinafter, referred to as the empty vector-harboring E. coli described in Japanese Patent Laid-Open No. 2017-216974) was also prepared as a comparative control. The preparation method followed Examples described in [0116] of Japanese Patent Laid-Open No. 2017-216974. The collected E. coli was suspended in a potassium phosphate buffer solution to obtain a potassium phosphate buffer solution of the E. coli.

<Microbial Cell Reaction>

In Examples 1 to 6, 500 μl of a reaction solution containing 5 mM α-guaiene, 5% (v/v) ethanol, 50 mM glucose, 0.5% (v/v) Tween® 80 as a surfactant, 1% (w/v) HP-β-CD, and each potassium phosphate buffer solution of the E. coli obtained under culture conditions 2 to 7 (50 mM, pH 7.5, OD₆₀₀: 30) was prepared and shaken at 30° C. for 6 hours to perform reaction.

In Comparative Example 1, reaction was performed in the same manner as in Examples 1 to 6 except that: the potassium phosphate buffer solution of the empty vector-harboring E. coli described in Japanese Patent Laid-Open No. 2017-216974 was used instead of the potassium phosphate buffer solution of the E. coli obtained under culture conditions 2 to 7; and no surfactant was added.

In Comparative Example 2, reaction was performed in the same manner as in Examples 1 to 6 except that: the potassium phosphate buffer solution of the E. coli obtained under culture condition 1 (no addition of Fe(III)-EDTA) was used instead of the potassium phosphate buffer solution of the E. coli obtained under culture conditions 2 to 7; and no surfactant was added.

In Comparative Example 3, reaction was performed in the same manner as in Examples 1 to 6 except that the potassium phosphate buffer solution of the E. coli obtained under culture condition 1 (no addition of Fe(III)-EDTA) was used instead of the potassium phosphate buffer solution of the E. coli obtained under culture conditions 2 to 7.

<Results of Microbial Cell Reaction>

After reaction, 1 ml of ethyl acetate was added for extraction. The extracts were subjected to high-performance liquid chromatography (HPLC) analysis and gas chromatography-mass spectrometry (GC/MS analysis). In this respect, the HPLC analysis was used to quantify products, and the GC/MS analysis was used to understand the behavior of the whole reaction product. The concentrations of rotundone as a product as well as a demethylated isomer formed at the same time with the rotundone were confirmed.

The HPLC analysis employed SHIMADZU PROMINENCE SERIES (manufactured by Shimadzu Corp.) and Wakosil-II 5C18 HG/HPLC column (manufactured by FUJIFILM Wako Pure Chemical Corp.). The analysis was conducted by using a sample injection volume of 10 μl, eluents A (ultrapure water) and B (acetonitrile), and a solution feed rate of 1 ml/min, and establishing a gradient program of 0 minutes (50% A+50% B)→10 minutes (0% A+100% B)→13 minutes (0% A+100% B)→14 minutes (50% A+50% B)→20 minutes (50% A and 50% B). The column temperature was set to 40° C. α-Guaiene was detected at a wavelength of 220 nm, and rotundone and a demethylated isomer were detected at a wavelength of 240 nm.

The GC/MS analysis employed Agilent 7890 Series GC System, 5977B Network Mass Selective Detector and HP-5 ms Ultra Inert/GC column (all manufactured by Agilent Technologies Inc.). The analysis was conducted using a sample injection volume of 1 μl, a split ratio of 10:1, He (1 ml/min) as a carrier gas, an injection port temperature of 250° C., an initial oven temperature of 40° C., an initial time of 1 min, a temperature increase rate of 15° C./min, and a final temperature of 300° C.

(Results of HPLC Analysis)

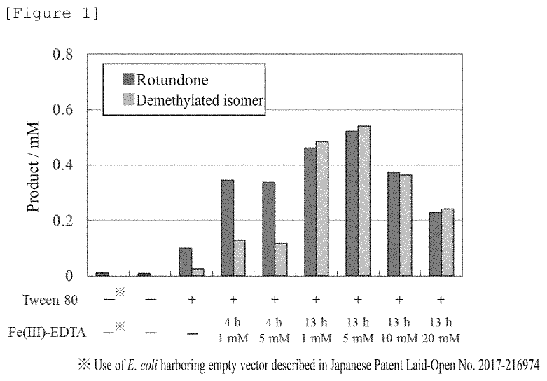

The results of HPLC analysis are shown in FIG. 1. In FIG. 1, the first to third columns from the left correspond to Comparative Examples 1 to 3, respectively, and the fourth to ninth columns from the left correspond to Examples 1 to 6, respectively. In the section Fe(III)-EDTA of FIG. 1, the “period from the start of culture after re-inoculation (of the preculture solution) to the addition of Fe(III)-EDTA (unit: h, i.e., hours)” is described in the upper part, and the “final concentration of Fe(III)-EDTA” is described in the lower part.

As seen from FIG. 1, in Comparative Example 1 using the empty vector-harboring E. coli described in Japanese Patent Laid-Open No. 2017-216974 without the addition of the trivalent iron compound (first column from the left), the concentration of rotundone was approximately 0.01 mM, and no demethylated isomer was detected.

In Comparative Example 2 using the non-recombinant E. coli without the addition of the trivalent iron compound (second column from the left), the concentration of rotundone was approximately 0.01 mM, and no demethylated isomer was detected.

On the other hand, as for series using the non-recombinant E. coli and using the surfactant, it was found in Examples 1 to 6 with the addition of the trivalent iron compound (fourth to ninth columns from the left) that the concentrations of rotundone and a demethylated isomer were markedly improved as compared with Comparative Example 3 without the addition of the trivalent iron compound (third column from the left).

From these results, it can be concluded that the microbial cell reaction which involved allowing non-recombinant E. coli-derived iron reductase to act in the presence of the trivalent iron compound contained in the non-recombinant E. coli cultured with the addition of the trivalent iron compound to sesquiterpene markedly promoted the synthesis reaction of rotundone and a demethylated isomer and was able to produce an oxygen adduct of sesquiterpene with a high yield and high purity.

When the addition of the trivalent iron compound (Fe(III)-EDTA) at the early stage and late stage of culture was compared among Examples 1 to 6, Examples 3 to 6 performed under the condition of addition of 5 mM at the late stage of culture (3 hours before the completion of culture, i.e., after a lapse of 13 hours from the start of culture after re-inoculation) resulted in the largest concentration of rotundone and also improved the concentration of a demethylated isomer. This is presumably because due to the addition of the trivalent iron compound at the early stage of culture, a chelate structure of a trivalent iron ion and EDTA was partially decomposed during subsequent culture, whereas the addition of the trivalent iron compound at the late stage of culture allowed the chelate structure of a trivalent iron ion and EDTA to be maintained until the time of reaction after bacterial collection. On the other hand, the demethylated isomer requires oxygen addition after demethylation and is difficult to synthesize. Therefore, its concentration is presumably decreased in the case of the addition of the trivalent iron compound at the early stage of culture.

The specific concentration of each product in FIG. 1 will be described below. As given below, the condition for Example 4 (culture condition 5, with addition of Fe(III)-EDTA at 5 mM in terms of a final concentration 3 hours before the completion of culture, i.e., after a lapse of 13 hours from the start of culture after re-inoculation) which produced the highest concentration of rotundone was found to have the improving effect on the amount of the product by approximately 50 times as compared with the condition for Comparative Example 1 or

In Comparative Example 1, rotundone: 0.01 mM, demethylated isomer: not detected.

In Comparative Example 2, rotundone: 0.01 mM, demethylated isomer: not detected.

In Comparative Example 3, rotundone: 0.09 mM, demethylated isomer: 0.02 mM.

In Example 1, rotundone: 0.34 mM, demethylated isomer: 0.13 mM.

In Example 2, rotundone: 0.33 mM, demethylated isomer: 0.11 mM.

In Example 3, rotundone: 0.46 mM, demethylated isomer: 0.48 mM.

In Example 4, rotundone: 0.52 mM, demethylated isomer: 0.53 mM.

In Example 5, rotundone: 0.37 mM, demethylated isomer: 0.36 mM.

In Example 6, rotundone: 0.22 mM, demethylated isomer: 0.24 mM.

(Results of GC/MS Analysis)

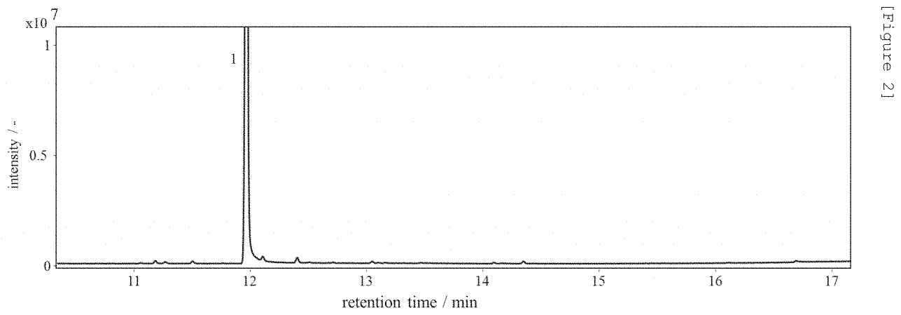

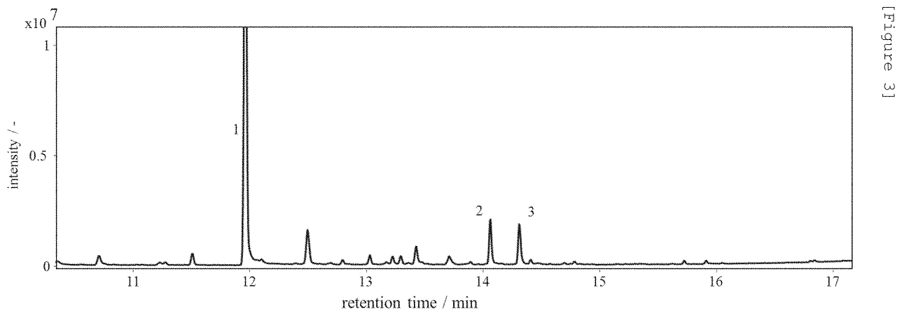

The results of GC/MS analysis are shown in FIGS. 2 and 3.

FIG. 2 shows a chromatogram obtained by GC/MS analysis in Comparative Example 2 without the addition of the trivalent iron compound (also without the addition of the surfactant). In FIG. 2, the peak of α-guaiene is peak 1.

FIG. 3 shows a chromatogram obtained by GC/MS analysis in Example 4 (culture condition 5, with addition of Fe(III)-EDTA at 5 mM in terms of a final concentration 3 hours before the completion of culture, i.e., after a lapse of 13 hours from the start of culture after re-inoculation). In FIG. 3, the peak of α-guaiene is peak 1, the peak of rotundone is peak 2 (retention time: 14.0 min), and a novel peak is peak 3 (retention time: 14.3 min).

<Isolation of Compound of Novel Peak>

The compound of peak 3, a novel peak, obtained in FIG. 3 was identified.

The reaction solution obtained in Example 4 was scraping-purified four times by silica gel thin-layer chromatography (hexane/ethyl acetate=8:1) to isolate each of 52 mg of rotundone and 49 mg of the compound of peak 3 (pale yellow oil, with UV absorption).

The isolated compound of peak 3 was subjected to ¹H NMR and ¹³C NMR analysis and GC/MS analysis. The obtained NMR data and GC/MS data will be given below.

¹H NMR (400 MHz, C₆D₆): δ=0.81 (3H, d, J=7.2 Hz), 1.27 (1H, m), 1.38 (1H, ddd, J=4.0, 4.0, 9.6 Hz), 1.45 (1H, m), 1.64 (3H, s), 1.63-1.69 (2H, m), 1.82 (1H, m), 1.98 (1H, m), 2.03-2.07 (3H, m), 2.11 (1H, m), 2.97 (1H, d, J=14.8 Hz), 4.75 (2H, d, J=15.6 Hz).

¹³C NMR (100 MHz, C₆D₆): δ=16.78, 20.70, 28.40, 29.87, 31.21, 32.42, 34.18, 36.72, 45.48, 109.20, 139.24, 150.68, 177.44, 207.23.

MS (EI, 70 eV), m/z (%): 204 (M+, 31), 189 (42), 161 (28), 148 (100), 133 (90), 119 (31), 105 (35), 91 (28), 79 (24).

From these results, the compound of the novel peak obtained in FIG. 3 was identified as a compound represented by the formula (1) ((4S,7R)-7-isopropenyl-4-methyl-3,4,5,6,7,8-hexahydro-1(2H)-azulenone; demethylated isomer of rotundone).

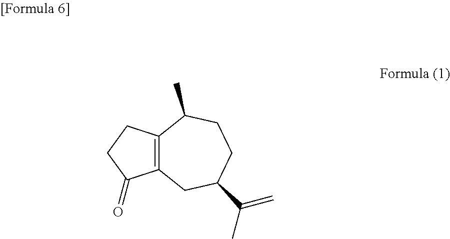

### [Examples 101 to 104 and Comparative Examples 101 to 104]: Microbial Cell-Free Reaction Using Purified Iron Reductase

In Examples 101 to 104, a rotundone synthesis test was conducted in vitro using purified iron reductase. The iron reductase used was YqjH in Example 101, Fpr in Example 102, NfsB in Example 103, and Fre in Example 104.

<Purification of Iron Reductase>

According to a conventional method, the gene of each enzyme was amplified by PCR with the genome of E. coli (Escherichia coli) BL21 (DE3) as a template, and digested with restriction enzymes. Each of the yqjH and fre genes was incorporated into vector pET-21a manufactured by Novagen/Merck KGaA, and each of the fpr and nfsB genes was incorporated into vector pET-28a manufactured by Novagen/Merck KGaA to prepare a plasmid vector. E. coli BL21 (DE3) was transformed with the prepared plasmid vector, cultured using LB medium and induced to express the enzyme of interest using IPTG, and then collected by centrifugation. The microbial cells thus collected were ultrasonicated and centrifuged again to prepare cell-free extracts. Then, a purified enzyme of each iron reductase was prepared by nickel affinity chromatography.

<Purified Enzyme Reaction>

In Example 101, 500 μl of a reaction solution containing 5 mM α-guaiene, 5% (v/v) ethanol, 5 mM NADPH, 5 mM Fe(III)-EDTA, 1% (v/v) Tween 80 and 0.3 mg/ml YqjH purified enzyme, and a HEPES-KOH buffer solution (50 mM, pH 7.0) was prepared and shaken at 30° C. for 2 hours to perform reaction. HEPES represents 4-(2-hydroxyethyl)-1-piperazineethanesulfonic acid.

In Example 102 or 103, the reaction was performed in the same manner as in Example 101 except that 0.1 mg/ml Fpr purified enzyme or NfsB purified enzyme was used instead of the 0.3 mg/ml YqjH purified enzyme.

In Example 104, the reaction was performed in the same manner as in Example 101 except that: 0.5 mg/ml Fre purified enzyme was used instead of the 0.3 mg/ml YqjH purified enzyme; and 1 mM FMN (flavin mononucleotide) was further added.

In Comparative Examples 101 to 104, the reaction was performed in the same manner as in Examples 101 to 104, respectively, except that no Fe(III)-EDTA was added.

<Results of Purified Enzyme Reaction>

After reaction, ethyl acetate extraction and HPLC and GC/MS analysis were performed in the same manner as in Examples 1 to 6.

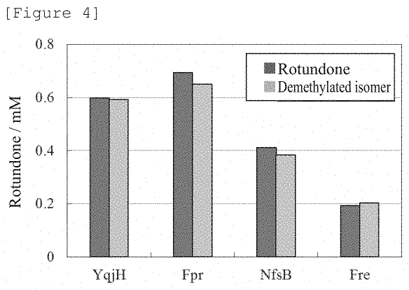

The results of HPLC analysis in Examples 101 to 104 are shown in FIG. 4. In FIG. 4, the first to fourth columns from the left correspond to Examples 101 to 104, respectively.

As is evident from FIG. 4, rotundone and a demethylated isomer were formed using any of the enzymes, though their concentrations (amounts of the products) differ among the enzymes. Each purified enzyme presumably caused coenzyme (NADPH, etc.)-dependent reduction of Fe³⁺ chelate and followed by the oxidation of α-guaiene into rotundone and a demethylated isomer.

On the other hand, in Comparative Examples 101 to 104 using a reaction system free from Fe(III)-EDTA, neither rotundone nor a demethylated isomer was formed.

Specific concentration of each product in FIG. 4 will be given below.

In Example 101 (using YqjH), rotundone: 0.60 mM, demethylated isomer: 0.59 mM.

In Example 102 (using Fpr), rotundone: 0.69 mM, demethylated isomer: 0.65 mM.

In Example 103 (using NfsB), rotundone: 0.41 mM, demethylated isomer: 0.38 mM.

In Example 104 (using Fre), rotundone: 0.19 mM, demethylated isomer: 0.20 mM.

### [Examples 111 to 112]: Influence of CD

In Example 111, the reaction was performed in the same manner as in Example 101 except that: the amount of the HEPES-KOH buffer solution was changed from 50 mM to 20 mM; the amount of the reaction solution was changed from 500 μl to 1 ml; and the reaction time was 20 hours.

In Example 112, the reaction was performed in the same manner as in Example 111 except that 1% (w/v) HP-β-CD was further added.

After reaction, each of the reaction solutions obtained by the reactions for 2 hours, 4 hours, 6 hours and 20 hours was subjected to ethyl acetate extraction and HPLC and GC/MS analysis in the same manner as in Examples 1 to 6. As a result, in Example 111, the concentration of rotundone in the reaction solutions after 4- to 20-hour reactions was approximately 0.62 mM. On the other hand, in Example 112 with the addition of HP-β-CD, the concentration of rotundone in the reaction solution after 4-hour reaction was merely more than 0.6 mM, whereas the concentration of rotundone in the reaction solution after 6-hour reaction was approximately 0.7 mM and the concentration of rotundone in the reaction solution after 20-hour reaction was 0.91 mM.

From these results, it is predicted that in Example 112, clathration using CD was able to reduce a loss of α-guaiene and improved the rate of conversion and the concentration of rotundone (amount of the product) as compared with Example 111.

### [Examples 113 and 114]: Influence of HP-γ-CD

In Example 113, the reaction was performed in the same manner as in Example 111 except that 1% (w/v) HP-γ-CD was further added.

In Example 114, the reaction was performed in the same manner as in Example 111 except that 1% (w/v) HP-β-CD and 1% (w/v) HP-γ-CD were further added.

As a result, in Example 113, the concentration of rotundone (amount of the product) was increased by approximately 6, as compared with Example 112 with the addition of HP-β-CD alone. In Example 114, the concentration of rotundone (amount of the product) was increased by approximately 13% as compared with Example 112.

### [Example 121]: Influence of Coenzyme Regeneration System on Reaction Using Purified Enzyme

In Example 121, 1 ml of a reaction solution containing 5 mM α-guaiene, 51 (v/v) ethanol, 1 mM NAD⁺, 5 mM Fe(III)-EDTA, 1% (v/v) Tween 80, 1% (w/v) HP-β-CD, 100 mM glucose, 0.1 U/ml GlcDH and 0.3 mg/ml YqjH purified enzyme, and a HEPES-KOH buffer solution (50 mM, pH 7.0) was prepared and reacted at 30° C. GlcDH represents glucose dehydrogenase derived from Bacillus sp., and 1 U (unit) means the amount of the enzyme that catalyzes the formation of 1 μmol NADH per minute.

After reaction, each of the reaction solutions obtained by the reactions for 2 hours, 4 hours, 6 hours and 20 hours was subjected to ethyl acetate extraction and HPLC and GC/MS analysis in the same manner as in Examples 1 to 6. The concentration of rotundone in the reaction solution after 2-hour reaction was approximately 0.9 mM, and the concentration of rotundone in the reaction solutions after 4- to 20-hour reactions was approximately 0.99 mM.

From comparison with Example 112 and Example 121 with the addition of HP-β-CD, coupling with the NADH regeneration system was found to improve a rotundone formation rate.

### [Examples 131 to 133]: Influence of Type of Fe³⁺ Ion on Reaction Using Fre Purified Enzyme

In Example 131, 500 μl of a reaction solution containing 5 mM α-guaiene, 5% (v/v) ethanol, 5 mM NADPH, 5 mM FeCl₃, 1% (v/v) Tween 80, 1 mM FMN and 1.2 mg/ml Fre purified enzyme, and a KH₂PO₄—K₂HPO₄ buffer solution (20 mM, pH 7.5) was prepared and reacted at 20° C. for 6 hours.

In Examples 132 and 133, the reaction was performed in the same manner as in Example 131 except that Fe(III)-EDTA or Fe(III)-dicitrate was used instead of FeCl₃.

After reaction, ethyl acetate extraction and HPLC and GC/MS analysis were performed in the same manner was in Examples 1 to 6.

As a result, in all of Examples 131 to 133, the concentration of rotundone (amount of the product) was approximately 0.20 mM. In short, reaction using the Fre purified enzyme had no significant difference in the amount of rotundone produced by the influence of the type of the Fe³⁺ ion. This is presumably because free flavin intervenes with the reduction of Fe³⁺.

### [Examples 141 to 143]: Influence of Type of Fe³⁺ Ion on Reaction Using YqjH Purified Enzyme

In Example 141, 500 μl of a reaction solution containing 5 mM α-guaiene, 5% (v/v) ethanol, 5 mM NADPH, 5 mM FeCl₃, 1% (v/v) Tween 80 and 0.4 mg/ml YqjH purified enzyme, and a KH₂PO₄—K₂HPO₄ buffer solution (20 mM, pH 7.5) was prepared and reacted at 20° C. for 6 hours.

In Examples 142 and 143, the reaction was performed in the same manner as in Example 141 except that Fe(III)-EDTA or Fe(III)-dicitrate was used instead of FeCl₃.

After reaction, ethyl acetate extraction and HPLC and GC/MS analysis were performed in the same manner as in Examples 1 to 6.

As a result, the concentration of rotundone (amount of the product) was approximately 0.05 mM in Example 141, approximately 0.58 mM in Example 142, and approximately 0.36 mM in Example 143. Accordingly, reaction using the YqjH purified enzyme was found to increase the amount of rotundone produced in the order of FeCl₃, Fe(III)-dicitrate and Fe(III)-EDTA.

When compared with the results of Examples 131 to 133 using the Fre purified enzyme, the results of Examples 141 to 143 using the YqjH purified enzyme were unexpected results. This is presumably due to the influence of the substrate specificity of YqjH.

### [Examples 151 to 153]: Influence of Addition Concentration of HP-β-CD

In Examples 151 to 153, α-guaiene was converted to rotundone in the same manner as in Example 112 except that the addition concentration of HP-β-CD in Example 112 was changed to 2% (w/v), 6% (w/v), or 12% (w/v). As a result, in all of Examples 151 to 153, the concentration of rotundone (amount of the product) was increased as compared with Example 112 having a HP-β-CD addition concentration of 1% (w/v). This amount of the product was approximately 117% in Example 151, approximately 140% in Example 152, and approximately 164% in Example 153, when that in Example 112 was defined as 100%.

### [Examples 154 to 156]: Influence of Addition Concentration of HP-γ-CD

In Examples 154 to 156, α-guaiene was converted to rotundone in the same manner as in Example 113 except that the addition concentration of HP-γ-CD in Example 113 was changed to 2% (w/v), 6 (w/v), or 12% (w/v). As a result, in all of Examples 154 to 156, the concentration of rotundone (amount of the product) was increased as compared with Example 113 having a HP-γ-CD addition concentration of 1% (w/v). This amount of the product was approximately 113% in Example 154, approximately 144% in Example 155, and approximately 165% in Example 156, when that in Example 113 was defined as 100-.

### [Example 157]: Influence of Addition Concentrations of HP-β-CD and HP-γ-CD

In Example 157, α-guaiene was converted to rotundone in the same manner as in Example 114 except that each of the addition concentrations of HP-β-CD and HP-γ-CD in Example 114 was changed to 12% (w/v). As a result, in Example 157, the concentration of rotundone (amount of the product) was increased as compared with Example 114 having HP-β-CD and HP-γ-CD addition concentrations of 1% (w/v) each. This amount of the product was approximately 136% in Example 157, when that in Example 114 was defined as 100%.

### [Examples 201 to 204]: Type of Sesquiterpene in Reaction Using Purified Enzyme

In Examples 201 to 204, the reaction was performed in the same manner as in Example 101 except that rotundol, (+)-valencene, β-caryophyllene or (+)-longifolene was used instead of α-guaiene.

After reaction, ethyl acetate extraction and HPLC and GC/MS analysis were performed in the same manner as in Examples 1 to 6.

As a result, the formation of oxygen adducts of rotundol, (+)-valencene, β-caryophyllene and (+)-longifolene was confirmed. The oxygen adduct of rotundol or (+)-valencene was obtained by oxygen addition to its allyl position, and epoxide of β-caryophyllene or (+)-longifolene was obtained by oxygen addition to its double bond.

### [Examples 301 to 306]: Type of Trivalent Iron Compound

Rotundone was produced by oxygen addition to α-guaiene using YqjH as iron reductase in the same manner as in Example 101 except that: iron complexes Fe(III)-EDTA (Example 301), Fe(III)—NTA (Example 302), Fe(III)-HEDTA (Example 303), Fe(III)-DTPA (Example 304), Fe(III)-tri-o-phenanthroline (Example 305), and Fe(III)-TPA (Example 306) were used as trivalent iron compounds; and the concentration of the HEPES-KOH buffer solution was changed to 200 mM.

As a result, the concentration of rotundone (amount of the product) in each Example was approximately 0.42 mM in Example 301, approximately 0.52 mM in Example 302, approximately 0.53 mM in Example 303, approximately 0.4 mM in Example 304, approximately 0.65 mM in Example 305, and approximately 1.18 mM in Example 306. These iron complexes employed oxygen and nitrogen, or nitrogen as a coordinating atom, and such complexes were confirmed to be able to efficiently produce rotundone.

These Examples demonstrated that the method for producing an oxygen adduct of sesquiterpene according to the present invention can efficiently produce an oxygen adduct of sesquiterpene and produces a high yield, high purity and a rapid reaction rate as compared with, particularly, conventional production methods.

### REFERENCE SIGNS LIST

1: Peak of α-guaiene
2: Peak of rotundone
3: Peak of the compound represented by the formula (1)

1. A method for producing an oxygen adduct of sesquiterpene, comprising: providing a system comprising an iron reduction unit; adding a trivalent iron compound to the system; and adding oxygen to sesquiterpene in the system comprising the iron reduction unit and the trivalent iron compound.

2. The method according to claim 1, wherein the iron reduction unit is iron reductase.

3. The method for producing an oxygen adduct of sesquiterpene according to claim 1, wherein, in the oxygen addition, oxygen is added selectively at an allyl position or a double bond of the sesquiterpene.

4. The method for producing an oxygen adduct of sesquiterpene according to claim 1, wherein
the sesquiterpene is α-guaiene, and
the oxygen adduct of sesquiterpene includes (−)-rotundone.

5. The method for producing an oxygen adduct of sesquiterpene according to claim 1, wherein, in the oxygen addition, selective demethylation and oxygen addition at an allyl position of the sesquiterpene are performed.

6. The method for producing an oxygen adduct of sesquiterpene according to claim 1, wherein
the sesquiterpene is α-guaiene,
the oxygen adduct of sesquiterpene includes (−)-rotundone and a demethylated isomer, and
the demethylated isomer is a compound represented by the following formula (1):

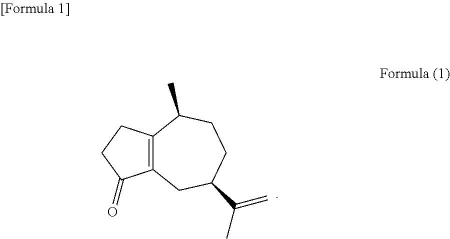

7. The method for producing an oxygen adduct of sesquiterpene according to claim 1, wherein a surfactant is added in the oxygen addition or prior thereto.

8. The method for producing an oxygen adduct of sesquiterpene according to claim 1, wherein
cyclodextrin is added in the oxygen addition or prior thereto, and
the cyclodextrin includes 2-hydroxypropyl-γ-cyclodextrin.

9. The method for producing an oxygen adduct of sesquiterpene according to claim 1, wherein
cyclodextrin is added in the oxygen addition step or prior thereto, and
the cyclodextrin includes at least one selected from α-cyclodextrin, β-cyclodextrin, γ-cyclodextrin, 2-hydroxypropyl-β-cyclodextrin and 2-hydroxypropyl-γ-cyclodextrin.

10. The method for producing an oxygen adduct of sesquiterpene according to claim 1, wherein
the oxygen addition is performed through microbial cell reaction, and
the iron reduction unit is iron reductase derived from the microbial cell.

11. The method for producing an oxygen adduct of sesquiterpene according to claim 10, wherein the addition of the trivalent iron compound to the system is performed before addition of the sesquiterpene.

12. The method for producing an oxygen adduct of sesquiterpene according to claim 10, further comprising
culturing the microbial cell, wherein
the trivalent iron compound is added in the culturing so that the trivalent iron compound is contained in the microbial cell.

13. The method for producing an oxygen adduct of sesquiterpene according to claim 12, wherein the trivalent iron compound is added at any time from 6 hours before the completion of the culturing to the completion of the culturing.

14. The method for producing an oxygen adduct of sesquiterpene according to claim 12, wherein the trivalent iron compound is added at 0.7 to 50 mM in the culturing.

15. The method for producing an oxygen adduct of sesquiterpene according to claim 10, wherein the microbial cell is non-recombinant E. coli.

16. The method for producing an oxygen adduct of sesquiterpene according to claim 10, wherein the microbial cell is a recombinant highly expressing at least one selected from the following (a) to (c):
(a) a protein having an amino acid sequence represented by any of SEQ ID NOs: 1 to 4;
(b) a protein which has an amino acid sequence having 90% or higher sequence identity to an amino acid sequence represented by any of SEQ ID NOs: 1 to 4, and has reductive activity against the trivalent iron compound; and
(c) a protein which has an amino acid sequence having 80% or higher sequence identity to an amino acid sequence represented by any of SEQ ID NOs: 1 to 4 with the conservative substitution of one or more amino acids from the amino acid sequence, and has reductive activity against the trivalent iron compound.

17. The method for producing an oxygen adduct of sesquiterpene according to claim 1, wherein the oxygen addition is performed through microbial cell-free reaction.

18. The method for producing an oxygen adduct of sesquiterpene according to claim 17, wherein
the iron reduction unit is iron reductase, and
the oxygen addition is performed using a purified enzyme of the iron reductase.

19. The method for producing an oxygen adduct of sesquiterpene according to claim 17, wherein the trivalent iron compound is added in the oxygen addition.

20. The method for producing an oxygen adduct of sesquiterpene according to claim 18, wherein the iron reductase is at least one selected from the following (a) to (c):
(a) a protein having an amino acid sequence represented by any of SEQ ID NOs: 1 to 4;
(b) a protein which has an amino acid sequence having 90% or higher sequence identity to an amino acid sequence represented by any of SEQ ID NOs: 1 to 4, and has reductive activity against the trivalent iron compound; and
(c) a protein which has an amino acid sequence having 80% or higher sequence identity to an amino acid sequence represented by any of SEQ ID NOs: 1 to 4 with the conservative substitution of one or more amino acids from the amino acid sequence, and has reductive activity against the trivalent iron compound.

21. The method for producing an oxygen adduct of sesquiterpene according to claim 2, wherein a coenzyme of the iron reductase is added in the oxygen addition.

22. The method for producing an oxygen adduct of sesquiterpene according to claim 21, wherein a regeneration system of the coenzyme is added in the oxygen addition to regenerate the coenzyme.

23. A compound represented by the following formula (1):

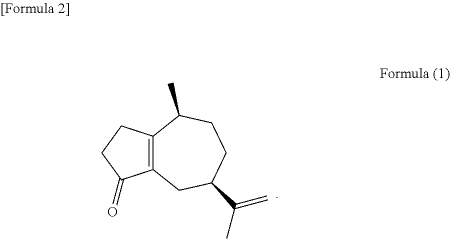

24. A composition comprising (−)-rotundone and a compound according to claim 23.


SEQUENCE LISTING (4 sequences)

SEQ ID NO: 1 - protein, 233 aa, Escherichia coli
MTTLSCKVTSVEAITDTVYRVRIVPDAAFSFRAGQYLMVVMDERDKRPFSMASTPDEKGFIELHIGASEINLYAKAVMDRILKDHQIVVDIPHGEAWLRDDEERPMILIAGGTGFSYARSILLTALARNPNRDITIYWGGREEQHLYDLCELEALSLKHPGLQVVPVVEQPEAGWRGRTGTVLTAVLQDHGTLAEHDIYIAGRFEMAKIARDLFCSERNAREDRLFGDAFAFI

SEQ ID NO: 2 - protein, 254 aa, Escherichia coli
MNNIPRYPQRVRNDLRFRELTVLRVERISAGFQRIVLGGEALDGFTSRGFDDHSKLFFPQPDAHFVPPTVTEEGIVWPEGPRPPSRDYTPLYDELRHELAIDFFIHDGGVASGWAMQAQPGDKLTVAGPRGSLVVPEDYAYQLYVCDESGMPALRRRLETLSKLAVKPQVSALVSVRDNACQDYLAHLDGFNIEWLAHDEQAVDARLAQMQIPADDYFIWITGEGKVVKNLSRRFEAEQYDPQRVRAAAYWHAK

SEQ ID NO: 3 - protein, 248 aa, Escherichia coli
MADWVTGKVTKVQNWTDALFSLTVHAPVHPFTAGQFTKLGLEIDGERVQRAYSYVNSPDNPDLEFYLVTVPDGKLSPRLAALKPGDEVQVVSEAAGFFVLDEVPDCETLWMLATGTAIGPYLSILQLGKDLDRFKNLVLVHAARYAADLSYLPLMQELEKRYEGKLRIQTVVSRETAAGSLTGRIPALIESGELESAIGLPMNKETSHVMLCGNPQMVRDTQQLLKETRQMTKHLRRRPGHMTAEHYW

SEQ ID NO: 4 - protein, 217 aa, Escherichia coli
MDIISVALKRHSTKAFDASKKLTPEQAEQIKTLLQYSPSSTNSQPWHFIVASTEEGKARVAKSAAGNYVFNERKILDASYVVVFCAKTAMDDAWLKLVVDQEDADGRFATPEAKAANDKGRKFFADMHRKDLHDDAEWMAKQVYLNVGNFLLGVAALGLDAVPIEGFDAAILDAEFGLKEKGYTSLVVVPVGHHSVEDFNATLPKSRLPQNITLTEV